(19) United States
(12) Patent Application Publication
(10) Pub. No.: US 2023/0100319 A1
(43) Pub. Date: Mar. 30, 2023

(54) A New Strain of Flammulina fennae and its molecular marker primers and molecular marker method

(71) Applicants: Guangdong Institute of Microbiology (Guangdong Detection Center of Microbiology), Guangzhou (CN); Guangdong Yuewei Biological Technology Co., Ltd., Zhaoqing (CN)

(72) Inventors: Qingping Wu, Guangzhou (CN); Huiping Hu, Guangzhou (CN); Yuanchao Liu, Guangzhou (CN); Yizhen Xie, Guangzhou (CN); Zhi Zhang, Guangzhou (CN); Xiaoxian Wu, Guangzhou (CN); Ao Wang, Guangzhou (CN); Lijun Zhuo, Guangzhou (CN); Chuan Shi, Guangzhou (CN)

(21) Appl. No.: 16/979,162

(22) PCT Filed: Dec. 2, 2019
(86) PCT No.: PCT/CN2019/119578

§ 371 Date: Sep. 8, 2020

(30) Foreign Application Priority Data
Sep. 11, 2019 (CN) 201910856184.4

(51) Int. Cl. A01H 15/00; C12Q 1/6895

(52) U.S. Cl. CPC A01H 15/00; C12Q 1/6895; C12Q 2600/158; C12R 2001/645

(57) ABSTRACT

The present invention relates to a new strain and its molecular marker primers and molecular marker method, in particular to a new strain of Flammulina fennae and its molecular marker primers and molecular marker method. The new strain was Flammulina fennae HMGIM-A151357. The deposit number was CCTCC NO: M 2019478. Compared with Flammulina velutipes on market, the Flammulina fennae of the present invention has a richer calcium content, a higher tolerant temperature, which was conducive to energy saving, and the mushroom cover was not easy to open the umbrella. The commercial properties were excellent, with the low content of cellulose. It was not easy to stuff teeth when eating, and the taste was better.

### TECHNICAL FIELD

The application relates to a new strain and its molecular marker primers and molecular marker method, in particular to a new strain of Flammulina fennae, and its molecular marker primers and molecular marker method.

### BACKGROUND

At present, the industry of edible and medicinal mushroom has developed rapidly. The yield of edible and medicinal mushroom in China reached more than 30 million tons, accounting for more than 75% of the world's total, with more than 20 million employees. This industry ranks fifth in the planting industry after grain, vegetables, fruits and cooking oil, exceeding cotton, tea and sericulture.

Now, with the flourishing development of the edible and medicinal mushroom industry, more and more rare species of edible and medicinal mushroom have been gradually discovered and applied. Many of rare varieties have been successfully domesticated, such as Dictyophora spp, Agrocybe cylindracea, Lyophyllum decastes and so on. However, a large number of wild mushrooms have not been studied for they have not been recognized. Among the 150,000 species of macro-fungi currently recorded, only about 80 kinds of wild edible and medicinal mushrooms were domesticated, and there are only about 20 varieties cultivated on a large scale. With the gradual increase of people's living standards, the requirements for quality of life are higher, and mushrooms are rich in various nutrients and functional ingredients, including fungal polysaccharides, triterpenoids, and sterols, etc., which are very important for human health, and has been paid more and more attention by people.

Flammulina velutipes is one of the widely cultivated edible mushrooms at present, and it is also a kind of edible and medicinal mushroom with high economic value. Flammulina velutipes contains a variety of nutrients, among which, the contents of 8 kinds of amino acids required for human growth are high, especially that the content of Lysine and Arginine are particularly rich, which can promote the growth and intellectual development of children, so it is often called “Intellectual Mushroom”. Flammulina velutipes contains flammulin, which has a significant anti-cancer function. Regular consumption of Flammulina velutipes can also prevent hypertension, treat hepatitis and gastrointestinal ulcers and other diseases. However, many strains of Flammulina velutipes currently used in China were from Japan or South Korea, and lack of strains with independent intellectual property rights. Flammulina fennae is a newly discovered and cultivated species with independent intellectual property rights. It has similar morphological characteristics and nutrition ingredients to Flammulina velutipes, but its development and application were insufficient.

### SUMMARY OF THE INVENTION

In view of the above shortcomings, the present invention provides a new strain of Flammulina fennae, and its molecular marker primers and molecular marker method.

The present invention provides a new strain of Flammulina fennae, which was isolated from the dead wood of broad-leaved trees in Laotudingzi Mountain, Huanren County, Benxi City, Liaoning Province. The original strain was obtained by tissue isolation and purification of the fruiting body, which was identified by morphological and molecular biology method as a new strain of Flammulina fennae, named Flammulina fennae HMGIM-A151357, and it has been deposited on Jun. 20, 2019 at the China Center for Type Culture Collection (CCTCC, Wuhan, China) with the deposit number: CCTCC NO: M 2019478.

The mycelium of the strain isolated by the inventor was vigorous, and the fruit body has been obtained through artificial domestication and cultivation, and the commercial properties were excellent. The whole genome sequencing and assembly of this strain have been completed, by comparing with the edible mushroom genome, it belongs to the Flammulina fennae Bas, Classified status precisely belongs to the Fungi, Basidiomycota, Agaricomycetes, Agaricales, Physalacriaceae, Flammulina.

The fruit body of Flammulina fennae of the present invention is yellow, with pale ochre yellow pileus, convex-parabolic, smooth, and gluey thin skin on the surface, slightly viscid, medium size, generally not extended to flat. Lamellae sinuate to adnexed, not equal length, moderately distant, white to pale cream. Stipe slenderness, slightly flexuose, cylindric, pale-yellow to brown, the color of the base of the stipe is much darker than the top of, with white and thin context.

The growth cycle is 60 days to 70 days, and the fruiting management time is 10 days to 13 days. The optimal temperature of mycelium is 22-24° C. The primordium forms at 15-16° C., and the optimal temperature of the fruiting body growth is 16-18° C. The mycelium grows moderately on the Comprehensive PDA medium, the mycelium is slightly dense, it is white to off-white in the early stage, appears yellowish in the later stage, and produces oidiospores.

Compared with Flammulina velutipes, the Flammulina fennae of the present invention has a richer calcium content, a higher tolerant temperature of the fruiting, which was conducive to energy saving. The mushroom cover was not easy to open the umbrella, the commercial properties were excellent, with the low content of the cellulose. It was not easy to stuff teeth when eating, and the taste was better.

### BRIEF DESCRIPTION OF DRAWINGS

FIG. 1 is the ITS sequencing result of the new strain of Example 1 of the invention.

FIG. 2 is the difference sites of Example 2.

FIG. 3 is PCR amplification electrophoretogram of the one pair of primers of Example 2.

FIG. 4 is PCR amplification electrophoretogram of another pair of primers of Example 2.

### DETAILED DESCRIPTION

The present invention provides a new strain of Flammulina fennae, obtained by field collection and isolation, which was identified as a new strain by molecular biology and morphology identification, and its whole genome has been sequenced and comparative analysis on the gene of Flammulina fennae strains which was widely used in the market were finished. Specific primers were designed to complete its molecular marker, realizing the protection of its own strains, and providing property rights protection for commercial applications.

A new strain of Flammulina fennae, isolated from on dead wood of Broadleaf tree in Laotudingzi Mountain, Huanren County, Benxi City, Liaoning Province. The original strain was obtained by tissue separation and purification of fruiting bodies, and was identified as Flammulina fennae, by morphology and molecular biology method. A new strain of Flammulina fennae, named as Flammulina fennae HMGIM-A151357, has been deposited on Jun. 20, 2019 at the China Center for Type Culture Collection (CCTCC, Wuhan, China), with the deposit number: CCTCC NO: M 2019478.

After sequencing and assembling the whole genome of this strain, through comparison analysis with the genes of strains of Flammulina velutipes that commonly used in the market, the differential gene sets were screened, specific primers and PCR amplify reaction were designed, then the amplified products were subjected to 2% agarose gel electrophoresis detected and sequenced. The results show that the two pairs of nucleic acid probe primers of the present invention can amplify specific products for Flammulina fennae of the present invention, but there is no product for the Flammulina velutipes, and the amplified products were sent to sequence. Then it was compared to the genome of Flammulina fennae, the similarity reached more than 98%, indicating that the two pairs of nucleic acid probe primers can specifically amplify the gene of Flammulina fennae of the present invention to distinguish the Flammulina velutipes on the market.

Molecular marker primers of the new strain of Flammulina velutipes (Flammulina fennae HMGIM-A151357), including FAA-F-1 and FAA-R-1, or FAA-F-2 and FAA-R-3;

wherein,

FAA-F-1:
5′-GCCCTCCCTTTACCAGTCTTGCTTCCGAT-3′
FAA-R-1:
5′-CTCCATCGGGTTGATGTCCGCAAA-3′
FAA-F-2:
5′-GCCCTCCCTTTACCAGTC-3′
FAA-R-3:
5′-ATTCCGCATCCTCAACCT-3′.

The molecular marker method of a new strain of Flammulina fennae (Flammulina fennae HMGIM-A151357), using primers FAA-F-1 and FAA-R-1, or FAA-F-2 and FAA-R-3 to perform PCR amplification. The specific amplified product was obtained, and the stain was identified as a new strain of Flammulina fennae (Flammulina fennae HMGIM-A151357).

The molecular marker method further includes PCR amplification of Flammulina velutipes using the pair of primers FAA -F-1 and FAA -R-1, or FAA -F-2 and FAA -R-3, showing no amplified product, indicating identification specificity of a new strain of Flammulina fennae (Flammulina fennae HMGIM-A151357).

The molecular marker method further includes performing control amplification using ITS1/ITS4 as a pair of primers, and the amplified products indicate the effectiveness of the PCR amplification reaction system.

The present invention will be further described below in conjunction with specific embodiments.

### EXAMPLE 1

### Molecular Biology Identification

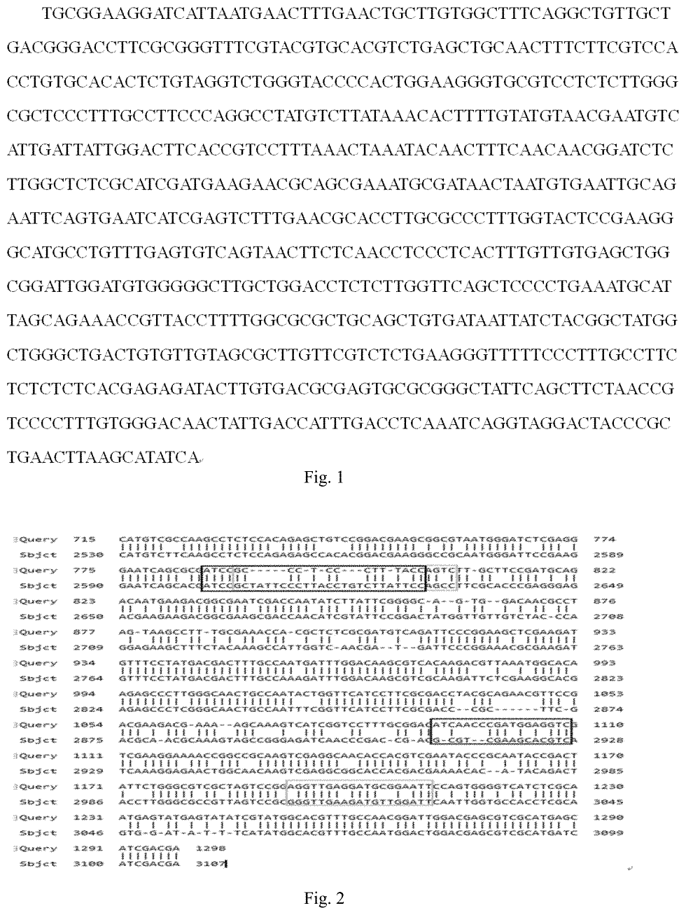

A new strain of Flammulina fennae, Flammulina fennae HMGIM-A151357, was isolated from the dead wood of broad-leaved trees of Laotudingzi Mountain, Huanren County, Benxi City, Liaoning Province, using Magen Bio HiPure Fungal DNA Mini Kit II to extract the genome DNA of mycelium. ITS1 (TCCGTAGGTGAACCTGCGG) and ITS4 (TCCTCCGCTTATTGATATGC) are used as primers to perform the ITS region PCR, and the amplified products are sent to Shanghai Meiji for Biosequencing to obtain the sequence of 760 bp, the sequence information as FIG. 1.

On the NCBI website, the blast program was used to compare and analyze the sequence, and the identification result was Flammulina fennae.

### EXAMPLE 2

### Artificial Cultivation Method

Inoculate the purified strain of Flammulina fennae into a 13 cm×25 cm polypropylene bag containing the original seed culture material. The composition of the original seed culture material was 98% sorghum grain, 2% calcium carbonate. Cultivate in the dark at 24-25° C. until the mycelium was full of the bag, then the original strain was obtained. Inoculate the original stain into the polypropylene mushroom bag containing cultivar culture material. The composition of the cultivar culture material were 50% cottonseed hull, 38% wood chips, 10% bran, and 2% calcium carbonate, placed in 24-25° C., cultivate until the bag was full of mycelium in a culture room with a relative humidity of about 70% in the dark. Then open the bag to promote buds and increase the humidity to about 95%. After water droplets appeared on the surface of the culture material, change the air twice a day to cultivate for about 4 days. Then cool down to 6° C. for cold stimulation, relative humidity of the air is about 85%, and illumination for 2 hours (daily), the processing time is about 4 days. Then raise the temperature to 18° C., the relative humidity of the air is about 85%, keep dark, and the carbon dioxide concentration is controlled to be less than 5%. When the young mushrooms grow to more than 10 cm and the caps begin to flatten, they can be harvested.

### EXAMPLE 3

### Specific Primers

By comparing the coding protein sequence (abbreviated as CDs sequence) of the Flammulina fennae with the CDs sequences of Flammulina velutipes, the differential gene was determined, and through screening, the gene of Flammulina fennae was determined to be obvious different from Flammulina velutipes gene, as shown in Table 1.

TABLE 1

| Query/ Flammulina fennae | Sbject/ Flammulina velutipes | Identities | E value |
|---|---|---|---|
| Gene13048 | 1c1\|GU169896.1-ADX07331.1-1 | 73.684 | 1.46E−48 |

The specific different sites are shown in FIG. 2.

According to the different sites in FIG. 2, two pairs of specific primers were designed, namely one pair of primers FAA-F-1/FAA-R-1, another pair of primers FAA-F-2/FAA-R-3, specific sequence as below:

Primer FAA-F-1:
5′-GCCCTCCCTTTACCAGTCTTGCTTCCGAT-3′
Primer FAA-R-1:
5′-CTCCATCGGGTTGATGTCCGCAAA-3′
Primer FAA-F-2:
5′-GCCCTCCCTTTACCAGTC-3′
Primer FAA-R-3:
5′-ATTCCGCATCCTCAACCT-3′

The above pairs of primers and corresponding PCR-amplified reaction system were used for identification. The PCR-amplified reaction system was 50 μL, which contains DNA polymerase mixed solution (Premix Taq™, RR003A) 25 μL, genomic DNA template of Flammulina fennae 5 μL, primers 5 μL each, ddH₂O 10 μL, the genomic DNA of Flammulina velutipes was as a control test, the PCR-amplified reaction procedure is shown in Table 2.

TABLE 2

| Temperature | | | | |
|---|---|---|---|---|
| FAA-F-1/ FAA-R-1 | FAA-F-2/ FAA-R-3 | Times | | Remarks |
| 95° C. | 95° C. | 5 | min | Pre-denaturation |
| 95° C. | 95° C. | 30 | s | 35Cycles |
| 68° C. | 51° C. | 30 | s | |
| 72° C. | 72° C. | 50 | s | |
| 72° C. | 72° C. | 10 | min | Cool down |

| | | | |
|---|---|---|---|
| 12° C. | 12° C. | ∞ | maintain |

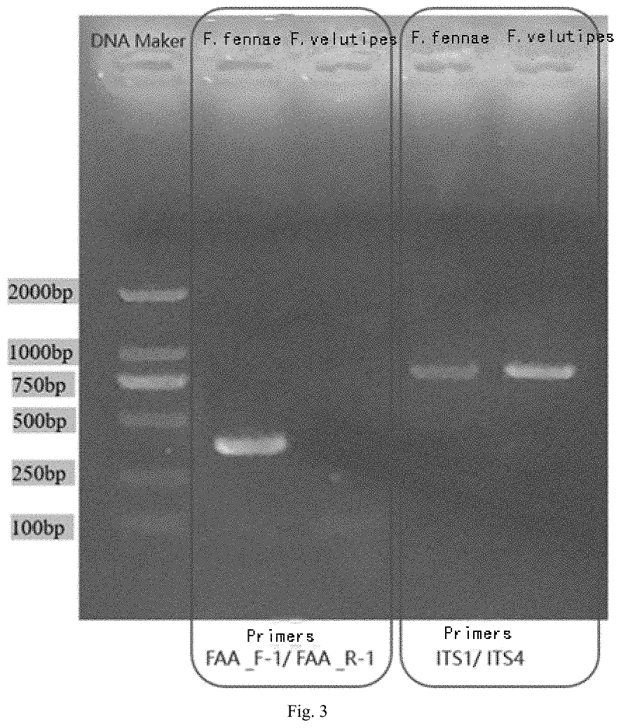

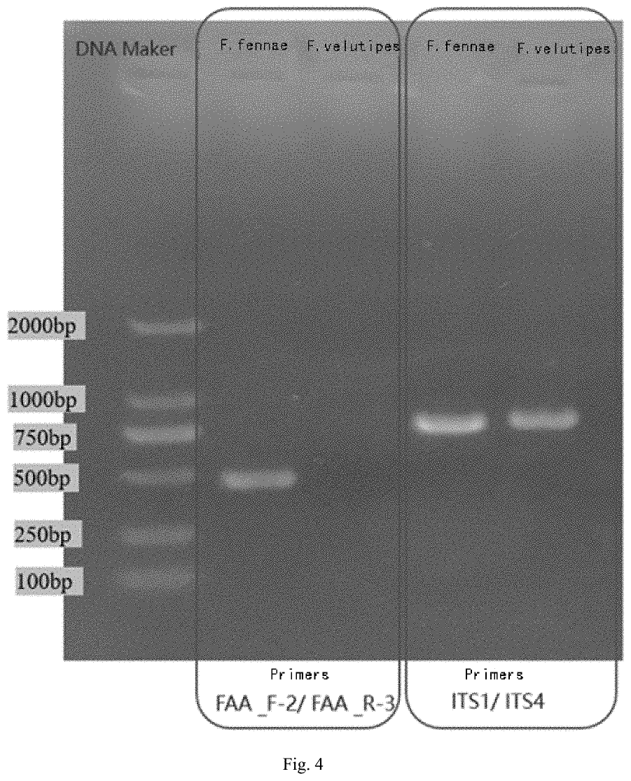

After the PCR reaction was completed, the products and DNA Marker were loaded on a 2% agarose gel, and electrophoresed at 120V for 25 minutes, and then the gel was taken to observed and photographed under fluorescence, as shown in FIG. 3 and FIG. 4.

As can be seen from FIG. 3 and FIG. 4, both Flammulina fennae of the present invention and Flammulina velutipes can amplify products under the primers of ITS1/ITS4. Therefore, the ITS primers cannot directly distinguish the two Flammulina spp from the amplified products, the primers of FAA-F-1/FAA-R-1, FAA-F-2/FAA-R-3 can specifically amplify the sequences of Flammulina fennae by PCR (300-310 bp and 420-430 bp respectively) , electrophoresis of PCR product can quickly distinguish this two kinds of Flammulina spp.

For the reason as above, the above two pairs of specific nucleotide primer sequences combined with conventional PCR and electrophoresis methods can quickly and accurately identify Flammulina fennae of the present invention to distinguish Flammulina velutipes on the market.

The above are only preferred specific embodiments of the present invention, but the scope of protection of the present invention is not limited to this. According to the technology of the present invention, scheme and its concept, any person skilled in the art in the technical field within the technical scope disclosed by the present invention can equivalently replaced or changed, which should be covered by the protection scope of the present invention.

1. A new strain of Flammulina fennae, characterized in that the new strain of Flammulina fennae is Flammulina fennae HMGIM-A151357, and the deposit number is CCTCC NO: M 2019478.

2. The molecular marker primers of the new strain of Flammulina fennae according to claim 1, characterized in that the molecular marker primers include FAA-F-1 and FAA-R-1;
FAA-F-1:
5′-GCCCTCCCTTTACCAGTCTTGCTTCCGAT-3′
FAA-R-1:
5′-CTCCATCGGGTTGATGTCCGCAAA-3′;

3. The molecular marker primers of the new strain of Flammulina fennae according to claim 1, characterized in that the molecular marker primers include FAA-F-2 and FAA-R-3;
FAA-F-2:
5′-GCCCTCCCTTTACCAGTC-3′
FAA-R-3:
5′-ATTCCGCATCCTCAACCT-3′.

4. The molecular marker method of a new strain of Flammulina fennae according to claim 1, characterized in that the primers are used for PCR amplification with FAA-F-1 and FAA-R-1, or FAA-F-2 and FAA-R-3, the specific amplified product was obtained, and the strain was identified as a new strain of Flammulina fennae (Flammulina fennae HMGIM-A151357).

5. The molecular marker method according to claim 4, characterized in that the molecular marker method further comprises that primers FAA-F-1 and FAA-R-1, or FAA-F-2 and FAA-R-3 are used for PCR amplification of Flammulina velutipes, which showed no amplified products, indicating the specificity of the molecular identification method of a new strain of Flammulina fennae (Flammulina fennae HMGIM-A151357).

6. The molecular marker method according to claim 4, characterized in that the molecular marker method further comprises that ITS1/ITS4 are used as primers for control amplification, and the amplified product indicates the effectiveness of the PCR amplification system.

7. The molecular marker method according to claim 5, characterized in that the molecular marker method further comprises that ITS1/ITS4 are used as primers for control amplification, and the amplified product indicates the effectiveness of the PCR amplification system.


SEQUENCE LISTING (7 sequences)

SEQ ID NO: 1 - DNA, 758 bp, Flammulina fennae
tgcggaaggatcattaatgaactttgaactgcttgtggctttcaggctgttgctgatgggaccttcgcgggtttcgtacgtgcacgtctgagctgcaactttcttcgtccacctgtgcacactctgtaggtctgggtaccccactggaagggtgcgtcctctcttgggcgctccctttgccttcccaggcctatgtcttataaacacttttgtatgtaacgaatgtcattgattattggacttcaccgtcctttaaactaaatacaactttcaacaacggatctcttggctctcgcatcgatgaagaacgcagcgaaatgcgataactaatgtgaattgcagaattcagtgaatcatcgagtctttgaacgcaccttgcgccctttggtactccgaagggcatgcctgtttgagtgtcagtaacttctcaacctccctcactttgttgtgagctggcggattggatgtgggggcttgctggacctctcttggttcagctcccctgaaatgcattagcagaaaccgttaccttttggcgcgctgcagctgtgataattatctacggctatggctgggctgactgtgttgtagcgcttgttcgtctctgaagggtttttccctttgccttctctctcacgagagatacctgtgacgcgagtgcgcgggctattcagcttctaaccgtcccctttgtgggacaactattgaccatttgacctcaaatcaggtaggactacccgctgaacttaagcatatca

SEQ ID NO: 2 - DNA, 19 bp, Artificial Sequence
  Artificial Sequence Universal primer for fungal ribosomal intergenic region (ITS1)
tccgtaggtgaacctgcgg

SEQ ID NO: 3 - DNA, 20 bp, Artificial Sequence
  Artificial Sequence Universal primer for fungal ribosomal intergenic region (ITS4)
tcctccgcttattgatatgc

SEQ ID NO: 4 - DNA, 29 bp, Artificial Sequence
  Molecular marker primers of the new strain (FAA -F-1)
gccctccctttaccagtcttgcttccgat

SEQ ID NO: 5 - DNA, 24 bp, Artificial Sequence
  Molecular marker primers of the new strain (FAA -R-1)
ctccatcgggttgatgtccgcaaa

SEQ ID NO: 6 - DNA, 18 bp, Artificial Sequence
  Molecular marker primers of the new strain (-FAA -F-2)
gccctccctttaccagtc

SEQ ID NO: 7 - DNA, 18 bp, Artificial Sequence
  Molecular marker primers of the new strain (FAA -R-3)
attccgcatcctcaacct